(19) United States
(12) Patent Application Publication
(10) Pub. No.: US 2025/0326734 A1
(43) Pub. Date: Oct. 23, 2025

(54) FLUORINATED DERIVATIVES OF METHOXYDIBENZO[B,F]OXEPIN AND METHOD FOR OBTAINING THEREOF

(71) Applicant: Politechnika Warszawska, Warszawa (PL)

(72) Inventors: Hanna Krawczyk, Warszawa (PL); Filip Borys, Warszawa (PL); Piotr Tobiasz, Jozefow (PL); Jakub Sobel, Klobuck (PL)

(73) Assignee: Politechnika Warszawska, Warszawa (PL)

(21) Appl. No.: 18/857,782

(22) PCT Filed: May 19, 2023
(86) PCT No.: PCT/PL2023/050036

§ 371 Date: Oct. 17, 2024

(30) Foreign Application Priority Data
May 20, 2022 (PL) P.441238

(51) Int. Cl. C07D 313/14; B01J 31/02

(52) U.S. Cl. CPC C07D 313/14; B01J 31/0237

(57) ABSTRACT

The invention provides fluorinated derivatives of methoxydibenzo[b,f]oxepin of general formula (1) and general formula (2) and a method for obtaining thereof in a catalysed reaction of fluoroazobenzene with methoxydibenzo[b,f]oxepin. The preferable catalyst of the reaction is triethylamine, and the compounds obtained may be useful as molecular switches in biological systems and photopharmacology.

The present invention provides fluorinated derivatives of methoxydibenzo[b,f]oxepin and a method for obtaining thereof in a catalysed reaction of fluoroazobenzene with methoxydibenzo[b,f]oxepin, which can be useful as molecular switches in biological systems and photopharmacology.

Molecular photoswitches are light-responsive compounds characterised by their ability to isomerise cis-trans double bonds as in, for example, azobenzene. They have found applications in the remote manipulation of biological systems [1], in smart materials [2], and molecular machines [3]. One of the more spectacular biological applications is the restoration of sight. During studies in blind mice, photochemical restoration of visual reactions was observed due to the action of an azo compound, which is a molecular switch that enables control of neuronal excitation [4]. In particular, their potential for application in photopharmacology [5] has generated considerable interest in recent years. The development of new molecular photoswitches is largely driven by the challenge of enabling the use of visible and red light or even near-infrared (NIR) light to work in ‘both directions’ [6]. This is especially relevant for medical applications, as red/NIR light enables access to deep (1 cm) tissues, by penetrating without the toxic effects caused by light higher energies [7].

Dibenzo[b,f]oxepins are an interesting class of compounds due to their numerous biological properties, e.g. anti-inflammatory [8], hypotensive [9], or anticancer [10]. Fluoroazobenzenes, on the other hand, are molecules that provide stable and bidirectional photoconversion and tissue compatibility features [11].

The objective of the invention is a fast, cheap and simple method for obtaining branched dibenzo[b,f] oxepin derivatives with fluoroazobenzenes characterised by good atomic economics.

The present invention provides fluorinated derivatives of methoxydibenzo[b,f]oxepin of general formula (1) and general formula (2):

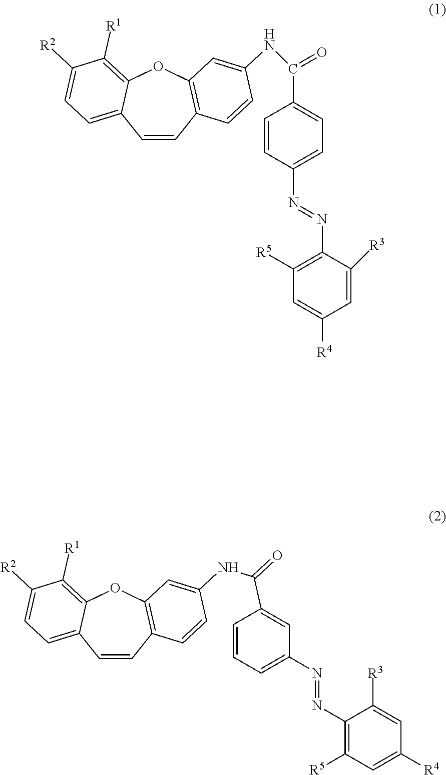

wherein:
R¹, R²=H, OMe;
R³, R⁴, R⁵=H, F.

The invention further provides a method for obtaining methoxydibenzo[b,f]oxepin derivatives of general formula (1) and general formula (2), which are preferably obtained by reacting substituted methoxydibenzo[b,f]oxepin of formula 3a-b with fluoroazobenzenes of formula 4a-d and 5a-d, carrying out the reaction in the presence of a solvent and a catalyst.

In the first step, thionyl chloride is added to fluoroazobenzene 4a-d and 5a-d, and thereafter the mixture is heated.

The reaction mixture is then evaporated to dryness, followed by dissolving several times in an aprotic organic solvent and evaporating again to dryness.

In the next step, methoxydibenzo[b,f]oxepin is dissolved in an aprotic organic solvent, a catalyst is added, and the residue from step (b) dissolved in an aprotic organic solvent, and the reaction mixture is then stirred.

Preferably, the ratio of fluorobenzene to thionyl chloride in step (a) is 1:50 mol/mol.

Preferably, the reaction in step (a) is heated to 80° C. for 30 min.

Preferably, the aprotic solvent in step (b) is methylene chloride.

Preferably, the aprotic solvent in step (c) is ethyl acetate.

Preferably, the catalyst in step (c) is triethylamine.

Preferably, the ratio of fluoroazobenzene to methoxydibenzo[b,f] oxepin is 1:1 mol/mol, more preferably 1:0.85 mol/mol.

Preferably, in step (c) a catalyst in a ratio of 25:1 mol/mol relative to fluoroazobenzene is used.

Preferably, the reaction in step (d) is stirred at 25° C. for 1 to 24 h.

The fast, cheap and simple method for obtaining fluorinated dibenzo[b,f] oxepin derivatives of general formula 1 and general formula 2, is characterized by good atomic economics. The method according to the invention may be useful in the synthesis of dibenzo[b,f] oxepin derivatives containing fluorine groups. Compounds of this type may find application as photo-switches in biological systems and photopharmacology.

### DESCRIPTION OF THE FIGURES

FIG. 1 shows fluoroazo derivatives of methoxydibenzo[b,f]oxepin of general formula (1) obtained by the method according to the invention.

FIG. 2 shows fluoroazo derivatives of methoxydibenzo[b,f]oxepin of general formula (2) obtained by the method according to the invention.

FIG. 3 shows the structure of methoxydibenzo[b,f]oxepin used to obtain azo derivatives of methoxydibenzo[b,f]oxepin in the method according to the invention.

FIG. 4 shows the general structure of fluoroazobenzene used as a substrate in the method for obtaining derivatives of general formula (1) according to the invention.

FIG. 5 shows the general structure of fluoroazobenzene used as a substrate in the method for obtaining derivatives of general formula (2) according to the invention.

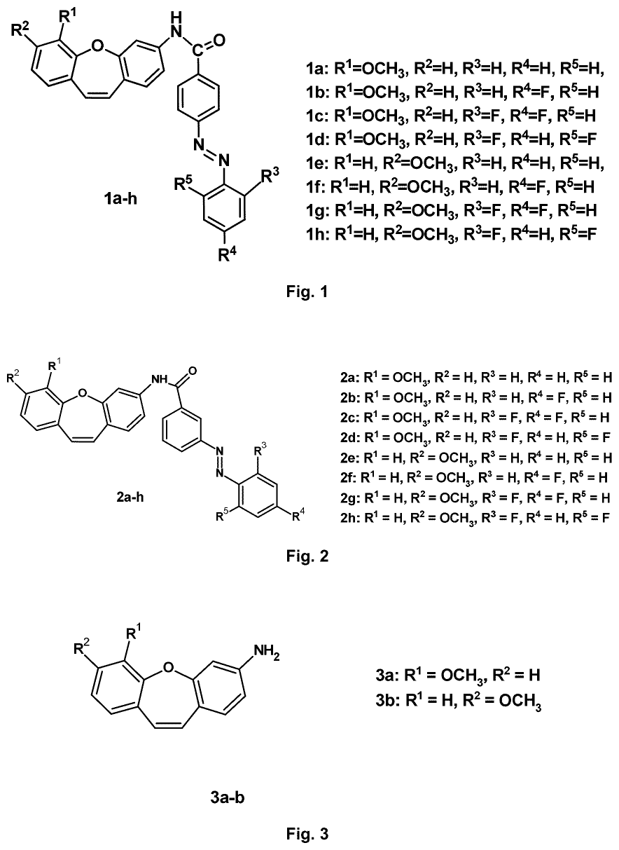

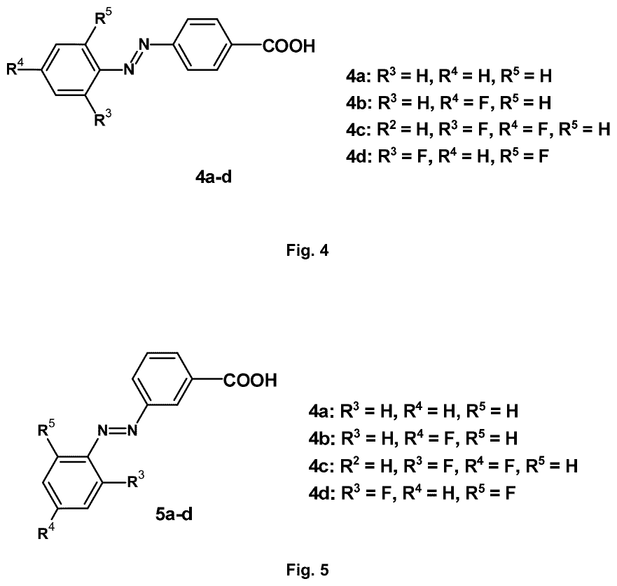

The following examples illustrate the invention without limiting it.

### IMPLEMENTATION EXAMPLE

### General Procedure for the Production of Compounds 1a-1h and Compounds 2a-2h

In a 5 ml round-bottom flask, 0.2 mmol of the corresponding fluoroazobenzene (4a-d/5a-d) and a magnetic stirring element were placed. Then 50 equivalents of thionyl chloride were added. The whole mixture was heated at 80° C. under a reflux condenser for about 30 minutes. The reaction mixture was evaporated to dryness on a rotary evaporator. Approximately 2 ml of DCM was added and evaporated again to get rid of residual SOCl₂. The operation was repeated twice. 0.85 equivalent of methoxydibenzo[b,f]oxepin (3a-b) was placed in a vial together with a magnetic stirring element and dissolved in 1 ml of ethyl acetate. The contents were stirred.

Then 0.25 mmol of triethylamine was added to the solution using an automatic pipette. The dry residue after evaporation of the thionyl chloride was dissolved in 1 ml of ethyl acetate and transferred to a vial with the contents being stirred. The system was closed with a stopper and the contents being stirred were left overnight.

After this time, the reaction mixture was analysed based on the position of the spots on a TLC plate in the hexane-ethyl acetate system at a volume ratio of 7:3. The mixture was evaporated and dissolved in a small amount of DCM and then purified on a chromatography column in the above system.

Sixteen derivatives (compounds of formulae 1a-h and 2a-h) were obtained as yellow-orange powders:
Compound (1a): yield: 46%, mp=215.5° C.
¹H NMR (500 MHz, DMSO-d₆, 298K): δ (ppm): Dibenzo[b,f]oxepin ring: 10.57 (1 H, s, NH), 7.79 (1 H, d, J_{H2,H4}=1.5 Hz, H₄), 7.66 (1 H, dd, J_{H1,H2}=8.5 Hz, H₂), 7.27 (1 H, d, H₁), 7.12-7.08 (2H, m, H₇, H₈), 6.83 (1 H, dd, J_{H8,H9}=6.5 Hz, J_{H7,H9}=3 Hz, H₉), 6.78 (1 H, AB spin system, d, J_{H10,H11}=11.5 Hz, H₁₀), 6.72 (1 H, spin system AB, d, H₁₁), 3.87 (3 H, s, OCH₃);
Azofluorobenzene ring: 8.18 (2 H, d, J_{H2.H3}=8.5 Hz, H₃), 8.02 (2 H, d, H₂), 7.95 (2 H, dd, J_{H2′,H3′}=8 Hz, J_{H2′,H4′}=2 Hz, H_{2′}), 7.65-7.61 (3 H, m, H_{3′}, H_{4′}).
¹³C NMR (125 MHz, DMSO-d₆, 298K): δ (ppm): 164.75, 164.53, 156.62, 156.56, 156.26, 153.53, 153.49, 151.92, 151.48, 151.45, 144.20, 144.17, 140.85, 136.74, 132.71, 132.11, 131.48, 131.47, 129.68, 129.57, 129.25, 129.17, 129.14, 128.92, 128.77, 128.69, 128.61, 127.59, 126.09, 125.95, 125.09, 125.06, 122.79, 122.40, 120.54, 120.51, 120.02, 119.52, 116.63, 116.53, 113.25, 113.16, 113.00, 112.97, 56.16, 56.13 (duplicated signals from atropoisomers).
HRMS (ESI): C₂₈H₂₁N₃O³⁺, calculated m/z: 447.15774; found m/z: 447.15778.
Compound (1b): yield: 76%, mp=242.5° C.

¹H NMR (500 MHz, DMSO-d₆, 298K): δ (ppm): Dibenzo[b,f]oxepin ring: 10.57 (1 H, s, NH), 7.78 (1 H, d, J_{H2.H4}=2.5 Hz, H₄), 7.66 (1 H, dd, J_{H1,H2}=8.5 Hz, H₂), 7.27 (1 H, d, H₁), 7.10-7.08 (2 H, m, H₇, H₈), 6.83 (1 H, dd, J_{H8.H9}=6.5 Hz, J_{H7.H9}=2.5 Hz, H₉), 6.78 (1 H, AB spin system, d, J_{H10,H11}=11.5 Hz, H₁₀), 6.72 (1 H, AB spin system, d, H₁₁), 3.87 (3 H, s, OCH₃); Azofluorobenzene ring: 8.18 (2 H, d, J_{H2,H3}=8.5 Hz, H₂), 8.03 (2 H, dd, J_{H2′,H3′}=8.5 Hz, J_{H2′,F}=5 Hz, H_{2′}), 8.01 (2 H, d, H₂), 7.47 (2 H, t, J_{H3′,F}=8.5 Hz, H_{3′}).
¹³C NMR (125 MHz, DMSO-d₆, 298K): δ (ppm): 165.15, 164.73, 163.15, 156.61, 156.09, 153.40, 151.47, 148.72, 148.70, 144.20, 140.84, 136.76, 131.48, 129.68, 129.25, 129.14, 128.77, 128.74, 128.70, 126.09, 125.26, 125.19, 125.09, 122.75, 122.68, 122.39, 120.54, 119.43, 116.68, 116.62, 116.54, 116.50, 115.95, 115.77, 113.24, 113.17, 113.00, 56.15 (duplicated signals from atropoisomers).
HRMS (ESI): C₂₈H₂₀FN₃O₃⁺, calculated m/z: 465.14832; found m/z: 465.14843.
Compound (1c): yield: 26%, mp=246° C.
¹H NMR (500 MHz, DMSO-d₆, 298K): δ (ppm): Dibenzo[b,f]oxepin ring: 10.58 (1 H, s, NH), 7.78 (1 H, d, J_{H2,H4}=2 Hz, H₄), 7.66 (1 H, dd, J_{H1,H2}=8.5 Hz, H₂), 7.27 (1 H, d, H₁), 7.09 (1 H, t, J_{H8,H9,7}=6.5 Hz, H₈), 7.08 (1 H, dd, J_{H7,H9}=2.5 Hz, H₇), 6.83 (1 H, dd, H₉), 6.78 (1 H, AB spin system, d, J_{H10.H11}=11.5 Hz, H₁₀), 6.72 (1 H, AB spin system, d, H₁₁), 3.87 (3 H, s, OCH₃); Azofluorobenzene ring: 8.18 (2 H, d, J_{H2,H3}=8.5 Hz, H₃), 8.01 (2 H, d, H₂), 7.87 (1 H, td, J_{H5′,H6′}=9 Hz, J_{H5′,F}=6.5 Hz H_{6′}), 7.62 (1 H, ddd, J_{H3′,F}=9 Hz, J_{H3′,H5′}=3 Hz, H_{3′}), 7.32-7.30 (1 H, m, H_{5′}).
¹³C NMR (125 MHz, DMSO-d₆, 298K): δ (ppm): 164.69, 164.45, 156.61, 156.56, 156.10, 153.54, 151.47, 151.45, 144.19, 144.17, 140.81, 140.79, 137.16, 133.73, 131.48, 131.47, 129.68, 129.66, 129.25, 129.21, 129.19, 128.78, 128.72, 126.12, 126.01, 125.09, 125.07, 122.61, 120.54, 120.52, 119.34, 119.25, 119.09, 116.64, 116.55, 113.25, 113.18, 113.00, 112.98, 112.79, 112.60, 105.97, 105.76, 105.57, 56.15, 56.13 (duplicated signals from atropoisomers).
HRMS (ESI): C₂₈H₁₉F₂N₃O₃⁺, calculated m/z: 483.13890; found m/z: 483.13884.
Compound (1d): yield: 16%, mp=230° C. ¹H NMR (500 MHz, DMSO-d₆, 298K): δ (ppm): Dibenzo[b,f]oxepin ring: 10.59 (1 H, s, NH), 7.78 (1 H, d, J_{H2,H4}=2 Hz, H₄), 7.65 (1 H, dd, J_{H1,H2}=8.5 Hz, H₂), 7.27 (1 H, d, H₁), 7.08 (1 H, t, J_{H8,H7,9}=6 Hz, H₈), 7.07 (1 H, dd, J_{H7,H9}=3 Hz, H₇), 6.82 (1 H, dd, H₉), 6.77 (1 H, AB spin system, d, J_{H10,H11}=11.5 Hz, H₁₀), 6.71 (1 H, AB spin system, d, H₁₁), 3.86 (3 H, s, OCH₃); Azofluorobenzene ring: 8.19 (2 H, d, J_{H2,H3}=8.5 Hz, H₃), 7.99 (2 H, d, H₂), 7.61 (1 H, tt, J_{H3′,H4′}=8.5 Hz, J_{H4′,F}=6 Hz, H_{4′}), 7.36 (2 H, t, J_{H3′,F}=9.5 Hz, H_{3′}).
¹³C NMR (125 MHz, DMSO-d₆, 298K): δ (ppm): 164.62, 164.39, 156.62, 156.56, 156.44, 155.96, 154.00, 153.90, 151.48, 151.45, 144.20, 144.17, 140.78, 140.76, 137.59, 134.61, 132.59, 131.48, 131.47, 130.23, 129.68, 129.66, 129.26, 129.24, 129.19, 128.87, 128.80, 128.74, 126.16, 126.05, 125.10, 125.07, 122.41, 120.54, 120.52, 118.25, 116.66, 116.56, 113.27, 113.25, 113.19, 113.09, 113.01, 112.98, 112.55, 112.37, 56.16, 56.13 (duplicated signals from atropoisomers).
HRMS (ESI): C₂₈H₁₉F₂N₃O₃⁺, calculated m/z: 483.13890; found m/z: 483.13901.
Compound (1e): yield: 30%, mp=168° C.
¹H NMR (500 MHz, DMSO-d₆, 298K): δ (ppm): Dibenzo[b,f]oxepin ring: 10.55 (s, ¹H, NH), 7.79 (1 H, d, J_{H2,H4}=2 Hz, H₄), 7.56 (1 H, dd, J_{H1,H2}=8.5 Hz, H₂), 7.23 (1 H, d, H₁), 7.18 (1 H, d, J_{H8,H9}=8.5 Hz, 6.80 (1 H, d, J_{H6,H8}=2.5 Hz, H₆), 6.77 (1 H, dd, H₈), 6.64 (1 H, AB spin system, d, J_{H10,H11}=11.5 Hz, H₁₀), 6.59 (1 H, AB spin system, d, H₁₁), 3.78 (3 H, s, OCH₃); Azofluorobenzene ring: 8.18 (2 H, d, J_{H2,H2}=8.5 Hz, H₂), 8.02 (2 H, d, H₃), 7.95 (2 H, dd, J_{H2′,H3′}=8.5 Hz, J_{H2′,H4′}=2 Hz, H_{2′}), 7.63-7.62 (3 H, m, H_{3′}, H_{4′}).
¹³C NMR (125 MHz, DMSO-d₆, 298K): δ (ppm): 164.69, 161.18, 157.31, 156.07, 153.55, 151.92, 140.62, 136.71, 132.11, 130.20, 129.57, 129.30, 129.08, 128.55, 127.20, 126.02, 122.92, 122.79, 122.43, 116.84, 112.97, 111.22, 106.68, 55.52.
HRMS (ESI): C₂₈H₂₁N₃O₃⁺, calculated m/z: 447.15774; found m/z: 447.15764.
Compound (1f): yield: 34%, mp=218° C.
¹H NMR (500 MHz, DMSO-d₆, 298K): δ (ppm): Dibenzo[b,f]oxepin ring: 10.55 (1 H, s, NH), 7.79 (1 H, d, J_{H2,H4}=2 Hz, H₄), 7.56 (1 H, dd, J_{H1,H2}=8.5 Hz, H₂), 7.23 (1 H, d, H₁), 7.18 (1 H, d, J_{H8,H9}=8.5 Hz, H₉), 6.80 (1 H, d, J_{H6,H8}=2.5 Hz, H₆), 6.77 (1 H, dd, H₈), 6.64 (1 H, AB spin system, d, J_{H10,H11}=11.5 Hz, H₁₀), 6.59 (1 H, AB spin system, d, H₁₁), 3.78 (3 H, s, OCH₃); Azofluorobenzene ring: 8.17 (2 H, d, J_{H2,H3}=8.5 Hz, H₃), 8.02 (2 H, dd, J_{H2′,H3′}=9 Hz, J_{H2′,F} =5 Hz, H_{2′}), 8.01 (2 H, d, H₂), 7.47 (2 H, t, J_{H3′,F}=9 Hz, H_{3′}).
¹³C NMR (125 MHz, DMSO-d₆, 298K): δ (ppm): 165.16, 164.67, 164.44, 163.16, 161.18, 161.15, 157.31, 157.28, 156.11, 156.07, 156.02, 153.42, 149.66, 149.64, 148.72, 148.69, 140.61, 140.50, 136.73, 136.68, 132.77, 130.20, 130.18, 129.30, 129.23, 129.08, 128.68, 128.55, 128.47, 127.20, 126.03, 125.89, 125.27, 125.20, 122.92, 122.75, 122.68, 122.43, 119.47, 116.84, 116.74, 116.69, 116.50, 115.95, 115.77, 112.97, 112.87, 111.21, 111.18, 106.68, 106.66, 55.52, 55.50 (duplicated signals from atropoisomers).
HRMS (ESI): C₂₈H₂₀FN₃O₃₊, calculated m/z: 465.14832; found m/z: 465.14824.
Compound (1g): yield: 23%, mp=215° C.
¹H NMR (500 MHz, DMSO-d₆, 298K): δ (ppm): Dibenzo[b,f]oxepin ring: 10.57 (1 H, s, NH), 7.79 (1 H, d, J_{H2,H4}=2 Hz, H₄), 7.56 (1 H, dd, J_{H1,H2}=8 Hz, H₂), 7.23 (1 H, d, H₁), 7.18 (1 H, d, J_{H8,H9}=8.5 Hz, H₉), 6.80 (1 H, d, J_{H6,H8}=2.5 Hz, H₆), 6.77 (1 H, dd, H₈), 6.64 (1 H, AB spin system, d, J_{H10,H11}=11.5 Hz, H₁₀), 6.59 (1 H, AB spin system, d, H₁₁), 3.78 (3 H, s, OCH₃); Azofluorobenzene ring 8.18 (2 H, d, J_{H2,H3}=8.5 Hz, H₃), 8.01 (2 H, d, H₂), 7.87 (1 H, td, J_{H5′,H6′}=8.5 Hz, J_{H6′,F}=6.5 Hz, H_{6′}), 7.62 (1 H, ddd, J_{H3′,F}=9 Hz, J_{H3′,H5′}=2.5 Hz, H_{3′}), 7.31-7.27 (1 H, m, H_{5′}).
¹³C NMR (125 MHz, DMSO-d₆, 298K): δ (ppm): 164.63, 161.19, 157.30, 157.28, 156.06, 153.58, 153.55, 140.58, 137.13, 130.20, 129.31, 129.24, 129.15, 128.67, 128.56, 127.20, 126.06, 125.94, 122.92, 122.65, 119.33, 119.25, 119.13, 116.85, 116.76, 112.99, 112.82, 112.63, 111.22, 106.68, 106.66, 105.98, 105.79, 105.57, 55.52, 55.51 (duplicated signals from atropoisomers).
HRMS (ESI): C₂₈H₁₉F₂N₃O₃⁺, calculated m/z: 483.13890; found m/z: 483.13864.
Compound (1h): yield: 39%, mp=175° C.
¹H NMR (500 MHz, DMSO-d₆, 298K): δ (ppm): Dibenzo[b,f]oxepin ring: 10.58 (1 H, s, NH), 7.79 (1 H, d, J_{H2,H4}=2 Hz, H₄), 7.56 (1 H, dd, J_{H1,H2}=8.5 Hz, H₂), 7.23 (1 H, d, H₁), 7.18 (1 H, d, J_{H8,H9}=8.5 Hz, H₉), 6.80 (1 H, d, J_{H6,H8}=2.5 Hz, H₆), 6.77 (1 H, dd, H₈), 6.64 (1 H, AB spin system, d, J_{H10,H11}=11.5 Hz, H₁₀), 6.59 (1 H, AB spin system, d, H₁₁), 3.78 (3 H, s, OCH₃); Azofluorobenzene ring: 8.19 (2 H, d, J_{H2,H3}=8.5 Hz, H₃), 8.00 (2 H, d, H₂), 7.62 (1 H, tt, J_{H3′,H4′}=8.5 Hz, J_{H4′,F}=6 Hz, H_{4′}), 7.37 (2 H, t, J_{H3′,F}=9 Hz, H_{3′}).
¹³C NMR (125 MHz, DMSO-d₆, 298K): δ (ppm): 164.55, 164.31, 161.19, 161.16, 157.31, 157.28, 156.44, 156.06, 156.01, 155.95, 154.00, 153.93, 150.91, 148.93, 140.55, 137.55, 134.57, 132.58, 130.20, 130.18, 129.31, 129.24, 129.18, 128.81, 128.57, 128.51, 127.19, 126.09, 125.97, 122.91, 122.90, 122.44, 118.29, 116.87, 116.75, 113.27, 113.24, 113.11, 113.09, 113.01, 112.89, 112.39, 111.22, 111.18, 106.68, 106.66, 55.52, 55.50 (duplicated signals from atropoisomers).
HRMS (ESI): C₂₈H₁₉F₂N₃O₃⁺, calculated m/z: 483.13890; found m/z: 483.13899.
Compound (2a): yield: 53%, mp=175° C. ¹H NMR (500 MHz, DMSO-d₆, 298K): δ (ppm): Dibenzo[b,f]oxepin ring: 10.62 (1 H, s, NH), 7.79 (1 H, d, J_{H2,H4}=2 Hz, H₄), 7.67 (1 H, dd, J_{H1,H2}=8.5 Hz, H₂), 7.28 (1 H, d, H₁), 7.09 (1 H, t, J_{H8,H7,9}=6.5 Hz, H₈), 7.08 (1 H, dd, J_{H7,H9}=2 Hz, H₇), 6.83 (1 H, dd, H₉), 6.78 (1 H, AB spin system, d, J_{H10,H11}=11.5 Hz, H₁₀), 6.72 (1 H, AB spin system, d, H₁₁), 3.87 (3 H, s, OCH₃); Azofluorobenzene ring: 8.47 (1 H, t, J_{H2,H4,6}=2 Hz, H₂), 8.15 (1 H, ddd, J_{H5,H6}=8 Hz, J_{H4,H6}=1 Hz, H₆), 8.10 (1 H, ddd, J_{H4,H5}=8 Hz, H₄), 7.95 (2 H, dd, J_{H2′,H3′}=8.5 Hz, J_{H2′,H4′}=1.5 Hz, H_{2′}), 7.77 (1 H, t, H₅), 7.66-7.61 (3 H, m, H_{3′}, H_{4′}).
¹³C NMR (125 MHz, DMSO-d₆, 298K): δ (ppm): 164.79, 164.52, 156.62, 156.60, 153.57, 153.32, 151.85, 151.77, 151.48, 151.45, 144.21, 144.19, 140.84, 140.72, 135.97, 135.29, 131.93, 131.48, 131.46, 130.59, 129.73, 129.69, 129.66, 129.58, 129.25, 128.95, 128.92, 128.77, 127.49, 126.39, 126.09, 126.07, 125.37, 125.09, 122.69, 122.05, 121.82, 120.55, 120.02, 119.93, 116.65, 116.54, 113.27, 113.14, 113.01, 56.16, 56.15 (duplicated signals from atropoisomers).
HRMS (ESI): C₂₈H₂₁N₃O₃H⁺, calculated m/z: 448.16557; found m/z: 448.16562.
Compound (2b): yield: 23%, mp=168.5° C.
¹H NMR (500 MHz, DMSO-d₆, 298K): δ (ppm): Dibenzo[b,f] oxepin ring: 10.62 (1 H, s, NH), 7.79 (1 H, d, J_{H2,H4}=2 Hz, H₄), 7.66 (1 H, dd, J_{H1,H2}=8.5 Hz, H₂), 7.27 (1 H, d, H₁), 7.12-7.08 (2 H, m, H₇, H₈), 6.83 (1 H, dd, J_{H8,H9}=6 Hz, J_{H7,H9}=3 Hz, H₉), 6.78 (1 H, AB spin system, d, J_{H10,H11}=11 Hz, H₁₀), 6.72 (1 H, AB spin system, d, H₁₁), 3.87 (3 H, s, OCH₃); Azofluorobenzene ring: 8.46 (1 H, t, J_{H2,H4,6} =1.5 Hz, H₂), 8.16-8.14 (1 H, m, H₆), 8.10-8.08 (1 H, m, H₄), 8.03 (2 H, dd, J_{H2,H3}=8.5 Hz, J_{H2,F}=5 Hz, H₂), 7.76 (1 H, t, J_{H5,H4,6} =8 Hz, H₅), 7.47 (2 H, t, J_{H3′,F}=8.5 Hz, H_{3′}).
¹³C NMR (125 MHz, DMSO-d₆, 298K): δ (ppm): 165.05, 164.77, 164.51, 163.06, 156.62, 156.60, 153.45, 151.65, 151.48, 151.46, 149.50, 149.47, 148.64, 148.61, 144.21, 140.83, 140.71, 135.98, 135.42, 131.48, 131.46, 130.60, 129.73, 129.69, 129.66, 129.25, 129.07, 128.77, 126.47, 126.09, 125.39, 125.15, 125.09, 125.07, 122.76, 122.69, 121.80, 120.55, 119.92, 116.68, 116.65, 116.55, 116.49, 115.95, 115.77, 113.26, 113.16, 113.01, 56.16, 56.15 (duplicated signals from atropoisomers).
HRMS (ESI): C₂₈H₂₀FN₃O₃H⁺, calculated m/z: 466.15615; found m/z: 466.15591.
Compound (2c): yield: 87%, mp=190.5° C.
¹H NMR (500 MHz, DMSO-d₆, 298K): δ (ppm): Dibenzo[b,f]oxepin ring: 10.63 (1 H, s, NH), 7.78 (1 H, d, J_{H2,H4}=2 Hz, H₄), 7.66 (1 H, dd, J_{H1,H2}=8.5 Hz, H₂), 7.27 (1 H, d, H₁), 7.10-7.09 (2 H, m, H₇,H₈), 6.83 (1 H, dd, J_{H8,H9}=6.5 Hz, J_{H7,H9}=3 Hz, H₉), 6.77 (1 H, AB spin system, d, J_{H10,H11}=11.5 Hz, H₁₀), 6.72 (1 H, AB spin system, d, H₁₁), 3.87 (3 H, s, OCH₃); Azofluorobenzene ring: 8.46 (1 H, t, J_{H2,H4,6} =2 Hz, H₂), 8.17 (1 H, ddd, J_{H5,H6}=8 Hz, J_{H4,H6}=1 Hz, H₆), 8.09 (1 H, ddd, J_{H4,H5}=8 Hz, H₄), 7.88 (1 H, td, J_{H5,H6}=8.5 Hz, J_{H6′,F}=6.5 Hz, H_{6′}), 7.77 (1 H, t, H₅), 7.62 (1 H, ddd, J_{H3′,F}=9 Hz, J_{H3′,H5′}=2.5 Hz, H₃), 7.31-7.27 (1 H, m, H_{5′}.).
¹³C NMR (125 MHz, DMSO-d₆, 298K): δ (ppm): 165.46, 164.68, 164.29, 163.45, 161.01, 158.95, 156.61, 156.59, 153.68, 151.84, 151.47, 151.45, 144.20, 144.18, 140.80, 140.66, 136.91, 136.04, 135.37, 131.48, 131.46, 131.01, 129.81, 129.68, 129.65, 129.25, 129.12, 128.78, 127.34, 126.12, 125.30, 125.09, 123.36, 122.27, 121.52, 120.54, 119.38, 116.65, 116.58, 113.27, 113.19, 113.00, 112.63, 112.10, 105.96, 105.75, 105.56, 105.19, 105.00, 104.79, 56.15 (duplicated signals from atropoisomers).
HRMS (ESI): C₂₈H₁₉F₂N₃O₃H₊, calculated m/z: 484.14672; found m/z: 484.14652.
Compound (2d): yield: 36%, mp=179.5° C.
¹H NMR (500 MHz, DMSO-d₆, 298K): δ (ppm): Dibenzo[b,f]oxepin ring: 10.64 (1 H, s, NH), 7.78 (1 H, d, J_{H2,H4}=2 Hz, H₄), 7.66 (1 H, dd, J_{H1,H2}=8.5 Hz, Hz), 7.27 (1 H, d, H₁), 7.10-7.08 (2 H, m, H₇,H₈), 6.83 (1 H, dd, J_{H8.H9}=6.5 Hz, J_{H7,H9}=3 Hz, H₉), 6.78 (1 H, AB spin system, d, J_{H10.H11}=11.5 Hz, H₁₀), 6.72 (1 H, AB spin system, d, H₁₁), 3.87 (3 H, s, OCH₃); Azofluorobenzene ring: 8.46 (1 H, t, J_{H2,H4,}=2 Hz, H₂), 8.21 (1 H, ddd, J_{H5,H6}=8 Hz, J_{H4,H6}=1.5 Hz, J_{H2,H6}=1 Hz, H₆), 8.07 (1 H, ddd, J_{H4,H5}=8 Hz, J_{H2,H4}=1 Hz, H₄), 7.79 (1 H, t, H₅), 7.61-7.59 (1 H, m, H_{4′}), 7.37 (2 H, t, J_{H3′,H4′}=9 Hz, J_{H3′,F}=9 Hz, H_{3′}).
¹³C NMR (125 MHz, DMSO-d₆, 298K): δ (ppm): 164.56, 164.06, 156.58, 155.94, 154.17, 153.89, 152.34, 151.48, 151.45, 150.97, 149.00, 146.02, 144.21, 144.19, 140.78, 140.70, 140.60, 136.06, 135.38, 132.34, 131.61, 131.45, 130.26, 130.19, 130.11, 129.91, 129.68, 129.65, 129.35, 129.26, 128.83, 128.80, 128.19, 126.17, 126.14, 125.10, 124.96, 122.14, 121.02, 120.55, 118.36, 116.68, 116.61, 113.30, 113.24, 113.21, 113.01, 112.51, 56.16 (duplicated signals from atropoisomers).
HRMS (ESI): C₂₈H₁₉F₂N₃O₃H⁺, calculated m/z: 484.14672; found m/z: 484.14667.
Compound (2e): yield: 80%, mp=193.5° C.
¹H NMR (500 MHz, DMSO-d₆, 298K): δ (ppm): Dibenzo[b,f]oxepin ring: 10.61 (1 H, s, NH), 7.80 (1 H, d, J_{H2,H4}=2 Hz, H₄), 7.57 (1 H, dd, J_{H1,H2}=8.5 Hz, H₂), 7.23 (1 H, d, H₁), 7.18 (1 H, d, J_{H8,H9}=8.5 Hz, H₉), 6.80 (1 H, d, J_{H6,H8}=2.5 Hz, H₆), 6.77 (1 H, dd, H₈), 6.64 (1 H, AB spin system, d, J_{H10,H11}=11.5 Hz, H₁₀), 6.59 (1 H, AB spin system, d, H₁₁), 3.78 (3 H, s, OCH₃); Azofluorobenzene ring: 8.47 (1 H, t, J_{H2,H4,6}=2 Hz, H₂), 8.15 (1 H, ddd, J_{H5,H6}=8 Hz, J_{H4,H6}=1 Hz, H₆), 8.10 (1 H, ddd, J_{H4,H5}=8 Hz, H₄), 7.95 (2 H, dd, J_{H2′,H3′}=8.5 Hz, J_{H2′,H4′}=1.5 Hz, H_{2′}), 7.77 (1 H, t, H₅), 7.65-7.60 (3 H, m, H_{3′}, H_{4′}).
¹³C NMR (125 MHz, DMSO-d₆, 298K): δ (ppm): 164.72, 164.44, 161.18, 157.31, 157.29, 156.06, 156.04, 153.59, 153.32, 151.84, 151.79, 140.62, 140.49, 135.94, 135.24, 131.94, 130.55, 130.20, 129.77, 129.58, 129.30, 129.28, 129.00, 128.93, 128.54, 127.50, 127.21, 127.17, 126.32, 126.02, 125.37, 122.93, 122.90, 122.69, 122.18, 121.77, 120.00, 119.78, 116.85, 116.72, 113.00, 112.86, 111.22, 106.68, 106.66, 55.52 (duplicated signals from atropoisomers).
HRMS (ESI): C₂₈H₂₁N₃O₃⁺, calculated m/z: 447.15774; found m/z: 447.15789.
Compound (2f): yield: 62%, mp=188.5° C.
¹H NMR (500 MHz, DMSO-d₆, 298K): δ (ppm): Dibenzo[b,f] oxepin ring: 10.61 (1 H, s, NH), 7.79 (1 H, d, J_{H2,H4}=2 Hz, H₄), 7.57 (1 H, dd, J_{H1,H2}=8 Hz, H₂), 7.23 (1 H, d, H₁), 7.18 (1 H, d, J_{H8,H9}=8.5 Hz, H₉), 6.80 (1 H, d, J_{H6,H8}=2.5 Hz, H₆), 6.77 (1 H, dd, H₈), 6.64 (1 H, AB spin system, d, J_{H10,H11}=11.5 Hz, H₁₀), 6.59 (1 H, AB spin system, d, H₁₁), 3.78 (3 H, s, OCH₃); Azofluorobenzene ring: 8.46 (1 H, t, J_{H2,H4,6}=1.5 Hz, H₂), 8.14 (1 H, ddd, J_{H5,H6}=8 Hz, J_{H4,H6}=1 Hz, H₆), 8.09 (1 H, ddd, J_{H4,H5}=8 Hz, H₄), 8.03 (2 H, dd, J_{H2′,H3′}=9 Hz, J_{H3,F}=5 Hz, H_{2′}), 7.77 (1 H, t, H₅), 7.47 (2 H, t, J_{H3′,F}=9 Hz, H_{3′}).
¹³C NMR (125 MHz, DMSO-d₆, 298K): δ (ppm): 165.05, 164.70, 164.42, 163.05, 161.64, 161.18, 159.68, 157.31, 157.29, 156.06, 156.04, 153.46, 151.66, 149.49, 149.47, 148.63, 148.60, 140.61, 140.48, 135.95, 135.37, 130.55, 130.20, 129.77, 129.29, 129.12, 128.54, 127.20, 127.17, 126.40, 126.02, 126.00, 125.38, 125.14, 125.07, 122.92, 122.90, 122.74, 122.67, 121.96, 121.76, 119.76, 116.84, 116.73, 116.67, 116.49, 115.95, 115.77, 112.99, 112.88, 111.21, 106.68, 106.66, 55.51, 54.89 (duplicated signals from atropoisomers).
HRMS (ESI): C₂₈H₂₀FN₃O₃⁺, calculated m/z: 465.14832; found m/z: 465.14853.
Compound (2g): yield: 89%, mp=169.5° C.
¹H NMR (500 MHz, DMSO-d₆, 298K): δ (ppm): Dibenzo[b,f]oxepin ring: 10.61 (1 H, s, NH), 7.79 (1 H, d, J_{H2,H4}=2 Hz, H₄), 7.56 (1 H, dd, J_{H1,H2}=8.5 Hz, H₂), 7.23 (1 H, d, H₁), 7.18 (1 H, d, J_{H8,H9}=8.5 Hz, H₉), 6.80 (1 H, d, J_{H6,H8}=2.5 Hz, H₆), 6.77 (1 H, dd, H₈), 6.64 (1 H, AB spin system, d, J_{H10,H11}=11.5 Hz, H₁₀), 6.59 (1 H, AB spin system, d, H₁₁), 3.78 (3 H, s, OCH₃); Azofluorobenzene ring: 8.46 (1 H, t, J_{H2,H4,6}=2 Hz, H₂), 8.17 (1 H, ddd, J_{H5,H6}=8 Hz, J_{H4,H6}=1 Hz, H₆), 8.09 (1 H, ddd, J_{H4,H5}=8 Hz, H₄), 7.87 (1 H, td, J_{H5′,H6′}=8.5 Hz, J_{H6′,F}=6 Hz, H_{6′}), 7.78 (1 H, t, H₅), 7.62 (1 H, ddd, J_{H3′,F}=9 Hz, J_{H3′,H5′}=2.5 Hz, H_{3′}), 7.27-7.31 (1 H, m, H_{5′}).
¹³C NMR (125 MHz, DMSO-d₆, 298K): δ (ppm): 165.37, 164.62, 164.21, 163.45, 161.18, 161.01, 158.95, 157.31, 157.29, 156.06, 156.04, 153.71, 151.86, 140.57, 140.43, 136.92, 136.02, 135.33, 130.95, 130.20, 129.85, 129.30, 129.28, 129.17, 128.55, 127.26, 127.19, 127.16, 126.05, 125.31, 123.38, 122.92, 122.89, 122.22, 121.66, 119.37, 119.29, 116.86, 116.77, 113.01, 112.92, 112.62, 112.11, 111.22, 106.68, 105.96, 105.77, 105.56, 105.19, 105.00, 104.79, 55.51 (duplicated signals from atropoisomers).
HRMS (ESI): C₂₈H₁₉F₂N₃O₃⁺, calculated m/z: 483.13890; found m/z: 483.13893.
Compound (2h): yield: 91%, mp=167.5° C.
¹H NMR (500 MHz, DMSO-d₆, 298K): δ (ppm): Dibenzo[b,f]oxepin ring: 10.63 (1 H, s, NH), 7.79 (1 H, d, J_{H2,H4}=2 Hz, H₄), 7.57 (1 H, dd, J_{H1,H2}=8.5 Hz, H₂), 7.23 (1 H, d, H₁), 7.18 (1 H, d, J_{H8,H9}=8.5 Hz, H₉), 6.80 (1 H, d, J_{H6,H8}=2.5 Hz, H₆), 6.76 (1 H, dd, H₈), 6.64 (1 H, AB spin system, d, J_{H10,H11}=11.5 Hz, H₁₀), 6.59 (1 H, AB spin system, d, H₁₁), 3.78 (3 H, s, OCH₃); Azofluorobenzene ring: 8.46 (1 H, t, J_{H2,H4,6}=1.5 Hz, H₂), 8.20 (1 H, ddd, J_{H5,H6}=8 Hz, J_{H4,H6}=1 Hz, H₆), 8.07 (1 H, ddd, J_{H4,H5}=8 Hz, H₄), 7.79 (1 H, t, H₅), 7.61 (1 H, tt, J_{H3′,H4′}=8.5 Hz, J_{H4′,F}=6 Hz, H_{4′}), 7.37 (2 H, t, J_{H3′,F}=9 Hz, H_{3′}).
¹³C NMR (125 MHz, DMSO-d₆, 298K): δ (ppm): 164.49, 163.98, 161.18, 157.31, 157.29, 156.06, 156.03, 154.21, 153.85, 152.35, 150.91, 148.98, 140.56, 140.37, 136.03, 135.33, 132.35, 131.55, 130.19, 129.94, 129.38, 129.29, 129.28, 128.59, 128.56, 128.09, 127.19, 127.15, 126.10, 126.07, 124.96, 122.92, 122.89, 122.09, 121.13, 118.21, 116.88, 116.80, 113.23, 113.04, 112.94, 112.50, 111.23, 106.68, 55.52 (duplicated signals from atropoisomers).
HRMS (ESI): C₂₈H₁₉F₂N₃O₃H⁺, calculated m/z: 484.14672; found m/z: 484.14640.

### References

1. W. A. Velema, W. Szymanski, B. L. Feringa, Journal of the American Chemical Society, 2014, 136(6), 2178-2191.
2. Z. Li, J. Liang, W. Xue, G. Liu, S. H. Liu, J. Yin, Supramolecular Chemistry, 2013, 26 (1), 54-65.
3. J. V. De Julián-Ortiz, B. Verdejo, V. Polo, E. Besalú, E. G.España, International Journal of Molecular Sciences, 2016, 17(7), 1131. doi.org/10.3390/ijms17071131.
4. M. Banghart, A. Mourot, D. Fortin, J. Yao, R. Kramer, D. Trauner, Angewandte Chemie, International Edition, 2009, 48(48), 9097-9101.
5. M. M. Lerch, M. J. Hansen, G. M. van Dam, W. Szymanski, B. L. Feringa, Angewandte Chemie, International Edition, 2016, 55(37), 10978-10999.
6. I. M. Welleman, M. W. H. Hoorens, B. L. Feringa, H. H. Boersma, W. Szymański, Chemical Science, 2020, 11, 11672-11691.
7. G. Alachouzos, A. M. Schulte, A. Mondal, W. Szymanski, B. L. Feringa, Angewandte Chemie, International Edition, 2022, e202201308 (1 of 8).
8. Y. Nagai, A. Irie, H. Nakamura, K. Hino, H. Uno, H. Nishimura, J. Med. Chem., 1982, 25, 1065.
9. R. Kiyama, T. Honma, K. Hayashi, M. Ogawa, M. Hara, M. Fujimoto, T. Fujishita, J. Med. Chem., 1995, 38, 2728.
10. Y. Bharath, B. Thirupathi, G. Ranjit, D. K. Mohapatra, Asian J. Org. Chem, 2013, 2(10), 848.
11. L. Agnetta, M. Bermudez, F. Riefolo, C. Matera, E. Claro, R. Messerer, T. Littmann, G. Wolber, U. Holzgrabe, M. Decker, J. Med. Chem. 2019, 62, 3009.

1. A fluorine derivative of methoxydibenzo[b,f]oxepin of general formula (1) and general formula (2): 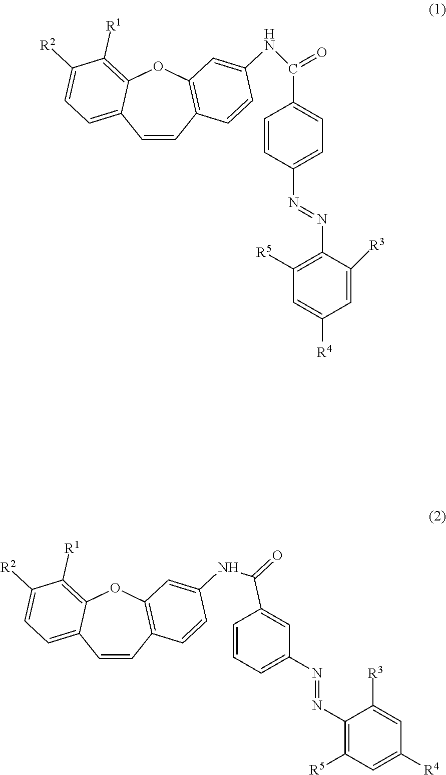
wherein:
R¹, R²=H or OMe; and
R³, R⁴, R⁵=H or F.

2. A method for obtaining the fluorine derivative as defined in claim 1, wherein the method comprises a reaction of a methoxydibenzo[b,f]oxepin of general formula (3): 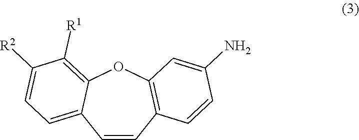
wherein:
R¹, R²=H or OMe;
with a fluoroazobenzene selected from the group of compounds of general formula (4) or (5): 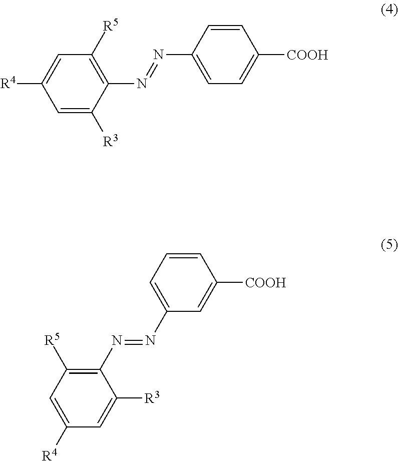
wherein:
R³, R⁴, R⁵=H or F;
in a solvent in the presence of a catalyst, comprising the following steps:
a) thionyl chloride is added to fluoroazobenzene, and thereafter the mixture is heated;
b) the reaction mixture is evaporated to dryness, then dissolved several times in an aprotic organic solvent and evaporated to dryness;
c) methoxydibenzo[b,f]oxepin is dissolved in an aprotic organic solvent, a catalyst is added, and the residue from step (b) is dissolved in the aprotic organic solvent; and
d) the reaction mixture is stirred.

3. The method according to claim 2, wherein the ratio of fluorobenzene to thionyl chloride in step (a) is 1:50 mol/mol.

4. The method according to claim 2, wherein the reaction mixture in step (a) is heated to 80° C. for 30 min.

5. The method according to claim 2, wherein the aprotic organic solvent in step (b) is methylene chloride.

6. The method according to claim 2, wherein the aprotic organic solvent in step (c) is ethyl acetate.

7. The method according to claim 2, wherein the catalyst in step (c) is triethylamine.

8. The method according to claim 2, wherein the ratio of fluoroazobenzene to methoxydibenzo[b,f]oxepin is 1:1 mol/mol, preferably 1:0.85 mol/mol.

9. The method according to claim 7, wherein the catalyst in step (c) is used in a ratio of 25:1 mol/mol relative to fluoroazobenzene.

10. The method according to claim 2, wherein the reaction mixture in step (d) is stirred at 25° C. for 1 h to 24 h.
